(19) United States
(12) Patent Application Publication
(10) Pub. No.: US 2003/0208142 A1
(43) Pub. Date: Nov. 6, 2003

(54) Vascular guidewire for magnetic resonance and /or fluoroscopy

(76) Inventors: Alexander C Boudewijn, Leek (NL); Wilfred P Heiner, Lemmer (NL)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/182,312

(22) PCT Filed: Jun. 12, 2001
(86) PCT No.: PCT/US01/18720

(51) Int. Cl. A61B 5/00

(52) U.S. Cl. 600/585

(57) ABSTRACT

A guidewire (10) compatible for use with magnetic resonance systems and/or x-ray fluoroscopy is made from non-metallic materials with a high specific electric impedance, preferably constructed primarily of polymers. An outer polymer tubular sheath (18) is provided that is sealed at a proximal end (24) and a distal end (26). One or more markers (16) may be provided in selected locations inside the polymer sheath (18). The markers (16) are magnetic resonance markers and/or radiopaque markers. The outer sheath (18) may be sheathed in a protective layer which is also non-metallic and tends to improve the physical characteristics of the guidewire.

### CROSS REFERENCE TO RELATED APPLICATION

This application claims priority of U.S. Provisional Patent Application No. 60/211,157, filed Jun. 12, 2000.

### BACKGROUND AND SUMMARY OF THE INVENTION

1. Technical Background

The present invention relates generally to intravascular medical devices, and more particularly to a medical guidewire for use with magnetic resonance systems and/or x-ray fluoroscopy. Such guidewires may be used in medical procedures for both diagnostic and interventional purposes.

2. Discussion

Guidewires are used in a wide variety of medical procedures, most often in conjunction with one or more other medical devices, including catheters. Such a catheter may be any of various types, such as vascular catheters including those for angiography or angioplasty, but should in any event generally have a tubular lumen or other guiding means for advancing or withdrawing over the guidewire.

With certain applications it is standard practice to design the catheter to make certain portions of the catheter visible inside the body of the patient by (i) x-ray imaging, which is called fluoroscopy, or (ii) magnetic resonance (MR) imaging. For the first type of imaging, fluoroscopy, often the tip of guiding catheters and diagnostic catheters are made from or are equipped with radiopaque materials or components, and become clearly visible on an x-ray screen. Examples of such catheters are given in commonly assigned U.S. Pat. Nos. 5,171,232 issued to Castillo et al. on Dec. 15, 1992, and 5,045,072 issued to Castillo et al. on Sep. 3, 1991, both of which are hereby incorporated herein by reference. A common such component is a metal marker band affixed to a metal device, usually made of a radiopaque metal including gold, platinum, tungsten, etc.

Structurally, guidewires are often long, thin metal wires that generally taper from one diameter at a proximal end which remains outside the body of the patient, to a smaller diameter at the opposite distal end. Specifically, vascular guidewires are often more than five feet long and have a maximum outer diameter of approximately 0.04 inches. The diameter of the main core wire is generally ground down precisely in a series of alternating tapering portions and constant diameter sections, to develop a selectively engineered flexibility profile along the length of the guidewire.

The guidewire distal tip is usually very flexible, both to avoid any possibility of vascular trauma and so that it can be selectively bent and twisted to advance it along a desired vascular path. Guidewires are designed to transmit this twisting force or torsion, so that as the guidewire proximal end is twisted or rotated, the guidewire distal tip tends to rotate through about the same angle. In addition, a floppy spring may often be affixed to the extreme distal tip of the guidewire for flexibility.

An example of a current guidewire is described in the commonly assigned U.S. Pat. No. 4,846,186, issued to Box et al. on Jul. 11, 1989, which is incorporated in this disclosure by reference. The Box et al. patent show a guidewire suitable for both diagnostic and therapeutic or interventional procedures, having a PTFE coating from the proximal end along a majority of its length. The core wire tapers in steps to a distal portion that is flattened and surrounded by a flexible spring, which is brazed to the extreme distal end of the core wire to form a rounded tip.

As the body of the patient is of course opaque, physicians commonly use fluoroscopy or x-ray video cameras to track the position of the guidewire and to construct real-time images of the patient's vasculature. The relative visibility and brightness of selected portions of the guidewire is a generally desirable feature, as described in the commonly assigned U.S. Pat. No. 5,259,393, issued to Corso, Jr. et al. on Nov. 9, 1993, and U.S. Pat. No. 5,267,574, issued to Viera et al. on Dec. 7, 1993. Both of these patents are incorporated in this disclosure by reference. In the Corso, Jr. et al. patent, the flexible spring at the guidewire distal tip is arranged to selectively control its brightness on an x-ray fluoroscope, or its radiopacity. Likewise, the Viera et al. patent discloses a plastic sleeve shrunk around an intermediate section of the guidewire, and several radiopaque marker bands.

In contrast to x-ray fluoroscopy, a second method of visualizing the patient is magnetic resonance imaging, referred to as MRI. Some medical fields, such as neurology, often use procedures which are performed under MRI instead of x-ray fluoroscopy. Accordingly, it is also desirable to image the anatomy and to track the position of intravascular devices, including catheters and guidewires, using magnetic resonance (MR) systems.

For these applications, it is desirable to make guidewires usable and compatible with MR techniques. However, a conventional metal guidewire may be too visible under MR, brightly washing out the screen and obscuring important features. This halo phenomenon is called an “artifact,” and renders the image useless. Another consideration with the use of a metal guidewire under MR is the induction of eddy currents in the metal, caused by distortion of the magnetic field. These eddy currents can generate heat and may increase the local temperature of the surrounding tissue and body fluids, thus possibly damaging the tissue or causing the blood to coagulate.

It is an object of the present invention to provide a guidewire having the desired physical features, including flexibility, torque transmission, and trackability while also avoiding the creation of undesirable artifacts in the MR image or the generation of heat, so that the guidewire is usable and compatible with both MR and x-ray fluoroscopy.

Another preferable object of the present invention may include providing a guidewire having at least portions that are visible under MR and/or fluoroscopy.

The present invention provides a guidewire compatible for use with magnetic resonance systems, made from a non-metallic material with a high specific electric impedance. Accordingly, this material will generally resist electrical eddy currents in the guidewire from being generated by variations in the high-frequency field. An acceptable class of materials is glass, which are all electrical insulators, and another type of materials is polymers. A guidewire having a major portion constructed of a glass or polymer material should therefore have advantages of not disturbing the MR field and images, as well as resisting the generation of heat.

The guidewire of the present invention further preferably includes one or more novel markers inside an outer sheath, for indicating positions of various portions of the guidewire or for measuring anatomy, etc. These markers are preferably capable of being visible under either or both magnetic resonance and x-ray fluoroscopy.

These and various other objects, advantages and features of the invention will become apparent from the following description and claims, when considered in conjunction with the appended drawings.

BRIEF DESCRIPTION OF THE DRAWINGS

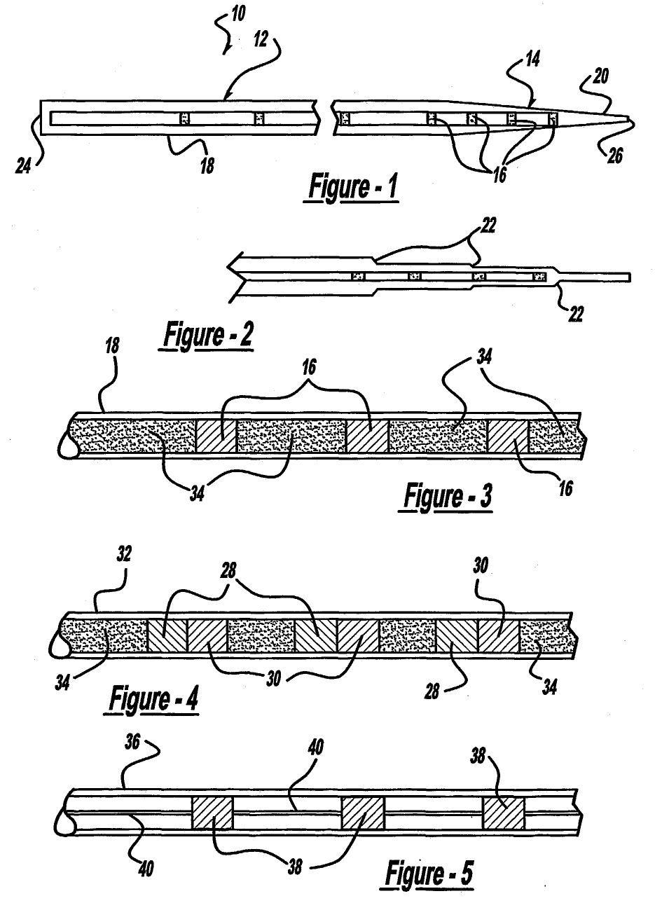

FIG. 1 is a longitudinal cross-sectional view of a guidewire for use with magnetic resonance and/or fluoroscopy systems, arranged according to an embodiment of the present invention; and

FIGS. 2-5 are partial longitudinal cross-sectional views of a portion of a guidewire according to other embodiments of the present invention.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

Referring to FIG. 1, a cross-sectional view of a guidewire according to a first preferred embodiment of the present invention is shown generally at 10. The medical guidewire 10 is intended for use in intravascular medical procedures involving the use of magnetic resonance and/or x-ray fluoroscopy systems, including both imaging and tracking of the guidewire's position within the body of the patient.

Guidewire 10 is constructed generally of a basic body 12 and a distal tip portion 14. The distal tip 14 of guidewire 10 includes several markers 16 embedded within the guidewire 10, which are more visible under an imaging system than the remainder of the guidewire 10.

In general, the guidewire designs of the present invention are preferably relatively long, thin, and flexible. As an example, vascular guidwires are often five feet long or more, and have an outer dimension of approximately 0.04 inches or less. A distal end of the guidewire is preferably even more flexible and floppy.

Additional desirable features include relatively high pull strength and column strength, steerability, and a tendency not to prolapse, etc. The guidewire further preferably transmits torque from a proximal end to a distal end, so a physician can effectively bend, turn, and steer the guidewire along a desired path.

The guidewire 10 is preferably made with a hollow outer tubular sheath 18. This tube 18 extends generally from a proximal end towards a distal end. The outer dimensions of the guidewire 10 preferably taper from the basic body 12 to the distal tip 14. This tapering feature may be accomplished in various ways, including the long basic tapering surface 20 illustrated in FIG. 1, and the step-wise tapering segments 22 shown in FIG. 2. Of course, the number and arrangement of tapers can be selected to provide desired performance in terms of strength and flexibility along the length of the distal segment 14 of the guidewire 10.

The outer polymer sheath 18 may be constructed from any of a variety of materials, including nylon, polyethylene, PET, polyurethane, HDPE, etc.

The outer sheath 18 is preferably sealed closed at both a proximal and distal end 24 and 26. These end seals may be formed in any conventional manner, including heat-sealing or preferably a UV-cured adhesive. This type of adhesive cures upon exposure to ultraviolet light. Accordingly, the polymer sheath 18 may preferably be transparent, to allow the glue to be exposed to the ultraviolet light after portions of the guidewire 10 are assembled.

Because a guidewire made mostly of polymers will not be visible with MR or radiation, markers 16 will be placed inside the lumen of the outer sheath 18. The markers will be placed at pre-defined positions. At the distal end 26 are preferably more markers at shorter intervals for positioning and measurement, and along the basic body 12 markers are placed at larger intervals for tracking up to a certain distance from the distal end, as shown in FIG. 1. The markers 16 may be made using various techniques, as described below.

Guidewire 10 preferably has spaced pairs of markers, as shown in FIG. 4, one of each pair being an MR marker 28 and one being an x-ray marker 30.

The markers 28 are visible under MR because their magnetic susceptibility differs to a controlled extent from the remainder of the guidewire and surrounding body tissue, thus distorting the uniformity of the magnetic resonance field and causing the magnetic field to become what is called “locally inhomogeneous.” The material of the markers 28 is selected specifically for his property, and acceptable materials include dysprosium oxide (Dy₂O₃). The markers 28 can be positioned by use of small volumes of dysprosium salt which will be positioned and fixed at predefined positions. This can be done by injecting the salt through a syringe needle or other long small tube, or by placing small lengths of dysprosium doped polymer, as a carrier, at the desired places.

The space between the markers can be filled with any suitable inert material 34, for instance, glue. Substantially the entire space inside the outer tube 18 is preferably filled with any suitable material, which may also be selected from amount various polymers including nylon, polyethylene, PET, polyurethane, HDPE, etc. The doped polymer parts can be fixated by glue at their predefined positions. The radiopaque markers 30 may be similarly made of any suitable radiopaque material, including gold, stainless steel, tungsten, or platinum. Preferably, the markers 30 may be made by injecting a small amount of tungsten salt at predefined positions. Of course, a small amount of polymer doped with tungsten or another radiopaque material may also be used.

As an alternative, when using salts, a mixed MR and x-ray radiation marker can be made by combining dysprosium salt and tungsten salt in the same marker.

Another way to add markers to the guidewire is to connect marker elements 38 at predefined positions on a thin polymer wire or glass fiber or core of the same material as the guidewire. The basic body 36 is illustrated in FIG. 5, and incorporates a relatively long, thin, and flexible core or glass body 40, which may be encased with a protective coating or cladding for improving the break strength of the glass body 40. The body 40 extends for substantially the length of the guidewire and is surrounded with a polymer sheath 36, which is adhered to the glass body 40 with a glue or some other adhesive.

The marker elements can be made of short pieces of dysprosium and/or tungsten doped polymer, as a carrier, or short rings of a MR compatible metal (gold, copper). The metal rings must be cut open to form a segmented non-continuous ring, to avoid activating the ring as a coil by a magnetic resonance field. The thin polymer wire or glass fiber with markers will be fixated into the lumen of the guidewire for example by using an adhesive.

The glass body and all of the guidewire components are preferably made of materials having a high specific electric impedance, such as polymers, fiberglass, silica or quartz.

The component may be provided with a cladding that adds strength to glass core, in that the coating allows any glass core or other components to be bent through a sharper turn or more tortuous path without breaking. A suitable material for the coating has been found to be polyimide.

An additional advantage of the design of the present invention is that the polymer sheath 18 can maintain the physical integrity of the guidewire, even if another component should unexpectedly break. Of course, the polymer sheath 18 may be provided with a lubricious coating, as generally known in the art.

An alternative embodiment of the present invention may be provided in which a number of reinforcing fibers has been embedded in the plastic sheath. Sheath may be shrunk around a bundle of fibers, or the sheath may be braided with the reinforcing fibers. Alternatively, fibers may be embedded in a polymer matrix, and surrounded by a coating. The reinforcing fibers may be of any suitable material, such as carbon, borium, aramide, or glass. The guidewire of the present invention may also be constructed of more than one glass core body, all of which may be clad as a unit with a single protective coating.

Use of the encapsulated markers makes portions of the guidewire visible for tracking and placement. In contrast to the more conventional markers disposed on the outside surface of a guidewire, which may affect profile dimensions, and which may act as a heating coil when constructed as metal outer rings, the present internal markers have various advantages. Internal markers preferably have substantially uniform size when viewed from any angle, producing a uniform indication without distortion.

The guidewire tube or outer sheath is preferably made out of one material, or it may be made out of several sections made of the same material that are adhered or melted together. The guidewire outer tube also defines a lumen for placement of MR and/or radiation compatible markers. As stated, magnetic resonance markers are preferably made of dysprosium salt, which is generally not visible or affected in an x-ray radiation environment. Likewise, x-ray radiation markers are preferably made of tungsten salt, which is generally not visible or affected in a magnetic resonance environment.

Markers may also be made of a combination of dysprosium and tungsten salts, which provides the advantage of a single marker that is both magnetic resonance and radiation visible and compatible. Markers can be positioned at precisely selected positions in the guidewire.

Also, cover tube or coating or cladding is necessary, although such a protective component may also be used if desired. The guidewire can be made in a wide range of diameters and length sizes. Material properties should be selected to affect properties of the guidewire, including flexibility, and torque.

It should be understood that an unlimited number of configurations for the present invention can be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A medical guidewire for use in intravascular medical procedures and having proximal and distal ends, comprising:
a relatively long, flexible tubular sheath extending for substantially the length of the guidewire, the sheath being made of a polymer material having a high specific electric impedance; with proximal and distal ends of the tubular sheath being sealed shut; a proximal portion of the guidewire having a constant outer dimension, and a distal portion of the guidewire transitioning to a smaller outer dimension at the distal tip of the guidewire; and
at least two markers positioned near the distal end of the guidewire, the markers all being surrounded by the tubular sheath, such that no portion of a marker is exposed to an exterior surface of the guidewire;
the guidewire being compatible with use in both magnetic resonance and x-ray fluoroscopy environments, wherein the markers are visible under magnetic resonance due to susceptibility-induced magnetic field inhomogeneity; each marker having substantially the same size when viewed under magnetic resonance from any angle; and wherein the guidewire is formed entirely of non-metallic materials.

2. The medical guidewire of claim 1, further comprising at least two radiopaque markers visible under x-ray fluoroscopy.

3. The medical guidewire of claim 2, wherein the MR markers and x-ray markers are arranged adjacent each other in pairs.

4. The medical guidewire of claim 1, wherein the MR markers are formed of dysprosium salt.

5. The medical guidewire of claim 2, wherein the x-ray markers are formed of tungsten salt.

6. The medical guidewire of claim 1, wherein the markers are dual-function markers, visible under both MR and x-ray fluoroscopy.

7. The medical guidewire of claim 1, wherein the markers are formed of polymer material doped with dysprosium salt.

8. A medical guidewire for use in intravascular medical procedures and having proximal and distal ends, comprising:
a relatively long, thin core extending for substantially the length of the guidewire, the core being made of a glass having a high specific electric impedance;
a polymer sheath surrounding the core;
at least one marker affixed to the core and positioned near a distal end of the guidewire, wherein the marker is visible under magnetic resonance due to susceptibility-induced magnetic field inhomogeneity; and the markers all being surrounded by the tubular sheath, such that no portion of a marker is exposed to an exterior surface of the guidewire; and
wherein the guidewire is formed entirely of non-metallic materials.

9. The medical guidewire of claim 8, further comprising a plurality of reinforcing fibers affixed to the core to enhance the flexibility and torsion characteristics of the guidewire.

10. The medical guidewire of claim 1, further comprising a plurality of reinforcing fibers affixed to the polymer sheath to enhance the flexibility and torsion characteristics of the guidewire.

11. The medical guidewire of claim 10, wherein the material of the reinforcing fibers is selected from the group consisting of carbon, borium, aramide, and glass.

12. The medical guidewire of claim 8, wherein the material of the core is selected from the group consisting of fiberglass, silica, and quartz.

13. The medical guidewire of claim 8 wherein a distal segment of the glass core tapers to a diameter at the distal end of the guidewire that is smaller than the diameter of a major portion of the core.

14. The medical guidewire of claim 1, wherein the material of the marker is Dysprosium Oxide (Dy₂O₃).

15. The medical guidewire of claim 1, wherein the distal tip of the guidewire is bent slightly, to facilitate the selective steering of the guidewire along a desired vascular path.

16. The medical guidewire of claim 8, wherein the core has a breaking strength, said core being covered with a cladding for increasing said breaking strength.

17. The medical guidewire of claim 1, wherein at least a portion of the polymer sheath has a hydrophilic coating.

18. The medical guidewire of claim 8, wherein the core is formed of a plurality of glass core strands.

19. A medical guidewire for use in intravascular medical procedures and having proximal and distal ends, comprising:
a relatively long, flexible tubular sheath extending for substantially the length of the guidewire, the sheath being made of a polymer material having a high specific electric impedance; with proximal and distal ends of the tubular sheath being sealed shut; a proximal portion of, the guidewire having a constant outer dimension, and a distal portion of the guidewire transitioning to a smaller outer dimension at the distal tip of the guidewire; and
at least one pair of two markers positioned near the distal end of the guidewire, the markers all being surrounded by the tubular sheath, such that no portion of a marker is exposed to an exterior surface of the guidewire;
the guidewire being compatible with use in both magnetic resonance and x-ray fluoroscopy environments, one of each pair of markers being visible under magnetic resonance, and the other of each pair of markers being visible under x-ray fluoroscopy; wherein the marker is visible under magnetic resonance due to susceptibility-induced magnetic field inhomogeneity; and wherein the guidewire is formed entirely of non-metallic materials.

20. The medical guidewire of claim 13, wherein the material of one of each pair of the markers is Dysprosium Oxide (Dy₂O₃).

21. The medical guidewire of claim 13, wherein the material of the other of each pair of markers is stainless steel.
